(19) United States
(12) Patent Application Publication
(10) Pub. No.: US 2025/0064579 A1
(43) Pub. Date: Feb. 27, 2025

(54) INTRAOCULAR LENS HAVING A MEDICAMENT RESERVOIR

(71) Applicant: Carl Zeiss Meditec AG, Jena (DE)

(72) Inventors: Niklas Damm, Oberkochen (DE); André Wolfstein, Oberkochen (DE)

(21) Appl. No.: 18/939,271

(22) Filed: Nov. 6, 2024

Related U.S. Application Data
(63) Continuation of application No. PCT/EP2023/062816, filed on May 12, 2023.

(30) Foreign Application Priority Data
May 20, 2022 (DE) 10 2022 112 803.2

(51) Int. Cl. A61F 2/16

(52) U.S. Cl. CPC A61F 2/1648; A61F 2002/1681

(57) ABSTRACT

An intraocular lens includes an optical body, which has an optical axis, and a haptic arm, which is attached to the optical body, wherein the haptic arm has a radial cutout formed in a side of the haptic arm, which side is arranged outwards in a radial direction with respect to the optical axis. The intraocular lens can have a medicament reservoir which has a medicament and a through-hole, in which the haptic arm is arranged, and which is arranged in the radial cutout, wherein the haptic arm has a radial projection which projects inwards in the radial direction from the rest of the haptic arm and which, in a circumferential direction with respect to the optical axis, is arranged in the same region as the radial cutout.

### CROSS-REFERENCE TO RELATED APPLICATIONS

This application is a continuation application of international patent application PCT/EP2023/062816, filed May 12, 2023, designating the United States and claiming priority from German application 10 2022 112 803.2, filed May 20, 2022, and the entire content of both applications is incorporated herein by reference.

### TECHNICAL FIELD

The disclosure relates to an intraocular lens having a medicament reservoir.

### BACKGROUND

When treating cataract of an eye, a natural lens is replaced by an artificial intraocular lens. To do this, only a small incision is usually made in the cornea of the eye, the incision being large enough to allow a tip of an injector to be inserted into the eye through the incision. After the incision has been made in the cornea, the natural lens of the eye is usually broken up by phacoemulsification and then aspirated out of the capsular bag of the eye. Thereafter, the intraocular lens is inserted into the eye by means of the injector.

After the cataract has been treated, medicaments such as antibiotics and/or anti-inflammatory agents are usually introduced into the eye. For example, a medicament reservoir can be attached to the intraocular lens, which medicament reservoir is inserted into the eye together with the intraocular lens. If the intraocular lens is inserted into the eye by means of the injector, the medicament reservoir should not detach from the intraocular lens. Moreover, the medicament reservoir should not be so bulky that the medicament reservoir leads to a non-centric arrangement of an optical body of the intraocular lens in the eye or causes structures for preventing aftercataract to be negatively affected. US 2022/0104936 A1 discloses an ophthalmic implant having an intraocular lens and haptics, and also medicament delivery devices that are attached to the haptics. US 2020/0405538 A1 discloses an ophthalmic device that can have an active or diagnostic agent.

### SUMMARY

It is an object of the disclosure to provide an intraocular lens which can receive a medicament reservoir, wherein the medicament reservoir, when received by the intraocular lens, does not detach from the intraocular lens and does not lead to decentration of an optical body of the intraocular lens in an eye.

An intraocular lens according to the disclosure includes an optical body, which has an optical axis, and a haptic arm, which is attached to the optical body, wherein the haptic arm has a radial cutout formed in a side of the haptic arm, which side is arranged outwards in a radial direction with respect to the optical axis. By means of the radial cutout, the intraocular lens is configured to receive a medicament reservoir. When the medicament reservoir is arranged in the radial cutout, the medicament reservoir is arranged particularly firmly on the haptic arm. There is therefore little possibility of the medicament reservoir detaching from the haptic arm, especially if the intraocular lens is injected via a tip of an injector into a capsular bag of an eye. Since the radial cutout is formed in that side of the haptic arm lying outwards in the radial direction, the medicament reservoir does not protrude, or protrudes only slightly, outwards from the haptic arm in the radial orientation. Since it is the side of the haptic arm lying outwards in the radial direction that contacts the capsular bag, it is thus possible to reduce or even avoid decentration of an optical body of the intraocular lens in the eye by the medicament reservoir, and structures for preventing aftercataract are not negatively affected.

According to various embodiments, the intraocular lens has a medicament reservoir which has a medicament and a through-hole, in which the haptic arm is arranged, and which is arranged in the radial cutout. It is particularly preferred that the medicament reservoir is recessed in the radial cutout. This means that the medicament reservoir does not protrude outwards from the haptic arm in the radial direction, as a result of which the decentration of the optical body can be particularly reliably avoided.

According to various embodiments, the medicament reservoir has a first end face and a second end face, which is arranged facing away from the first end face, wherein the through-hole is limited by the first end face and the second end face, and has a first longitudinal end in a region of the first end face and has a second longitudinal end in a region of the second end face, wherein the medicament reservoir has a displacement direction, which is directed from the first longitudinal end to the second longitudinal end. In order to introduce the medicament reservoir into the radial cutout, the haptic arm can have a longitudinal end which is arranged facing away from the optical body and which can be introduced into the through-hole. Subsequently, by displacement of the medicament reservoir in the displacement direction, the medicament reservoir can be displaced until the medicament reservoir enters the radial cutout. It is conceivable that the displacement direction is arranged parallel to a normal of the first end face and/or parallel to a normal of the second end face. The displacement direction preferably lies in a plane whose normal is arranged parallel to the optical axis. Alternatively, the displacement direction is preferably arranged parallel to the optical axis.

According to various embodiments, the haptic arm has a first axial cutout, which is formed in a first side of the haptic arm, which side is arranged outwards in an axial direction with respect to the optical axis. It is particularly preferred that the medicament reservoir is arranged in the first axial cutout. The medicament reservoir can thus be arranged even more firmly on the haptic arm, and protrusion of the medicament reservoir from the haptic arm can be reduced still further, as a result of which a possibility of the medicament reservoir detaching from the haptic arm during injection of the intraocular lens can be reduced still further. According to various embodiments, the first axial cutout directly adjoins the radial cutout.

According to various embodiments, the medicament reservoir has a first medicament reservoir cutout, which communicates with the through-hole, and a first web, which limits the first medicament reservoir cutout, wherein the first web is arranged in the first axial cutout and the haptic arm is arranged in the first medicament reservoir cutout. In particular, the first web can be recessed in the first axial cutout. Thus, the first web advantageously does not protrude in the axial direction from the haptic arm.

According to various embodiments, the haptic arm has a second axial cutout, which is arranged offset from the first axial cutout in a circumferential direction with respect to the optical axis and is formed in a second side of the haptic arm, which side is arranged outwards counter to the axial direction and is arranged facing away from the first side. It is particularly preferred that the medicament reservoir is arranged in the second axial cutout. The medicament reservoir can thus be arranged even more firmly on the haptic arm, and protrusion of the medicament reservoir from the haptic arm can be reduced still further, as a result of which a possibility of the medicament reservoir detaching from the haptic arm during injection of the intraocular lens can be reduced still further. According to various embodiments, the second axial cutout directly adjoins the radial cutout.

According to various embodiments, the medicament reservoir has a second medicament reservoir cutout, which communicates with the through-hole, and a second web, which limits the second medicament reservoir cutout, wherein the second web is arranged in the second axial cutout and the haptic arm is arranged in the second medicament reservoir cutout. In particular, the second web can be recessed in the second axial cutout. Thus, the second web advantageously does not protrude from the haptic arm counter to the axial direction.

According to various embodiments, the medicament reservoir, seen in the radial direction, is arranged flush with the haptic arm. Thus, detachment of the medicament reservoir from the haptic arm during the injection of the intraocular lens can be particularly reliably avoided. It is also readily possible, if folding the intraocular lens before injection of the intraocular lens, to transfer the haptic arm onto the optical body and fold the optical body around the haptic arm.

According to various embodiments, the second axial cutout is arranged spaced apart from the first axial cutout in the circumferential direction. Thus, severe weakening of the haptic arm by the first axial cutout and the second axial cutout can be avoided; weakening of the haptic arm can lead to the haptic arm tearing, especially when the haptic arm is inserted into the capsular bag via the tip of the injector.

According to the disclosure, the haptic arm has a radial projection which projects inwards in the radial direction from the rest of the haptic arm and which, in a circumferential direction with respect to the optical axis, is arranged in the same region as the radial cutout. This can prevent severe weakening of the haptic arm.

According to various embodiments, the haptic arm, in the region of the radial cutout, has a tensile strength of at least 0.25 N.

According to various embodiments, the haptic arm has a curved configuration. The haptic arm particularly preferably has a C-shaped or J-shaped configuration.

According to various embodiments, the haptic arm has a further radial cutout, which is formed in that side of the haptic arm arranged outwards in the radial direction. Moreover, the haptic arm can have a third axial cutout, which is formed in the first side of the haptic arm. In addition, it is conceivable that the haptic arm has a fourth axial cutout, which is formed in the second side of the haptic arm. It is particularly preferred that the intraocular lens has a further medicament reservoir, which has the medicament and a through-hole through which the haptic arm extends. The further medicament reservoir can be arranged in the further radial cutout and in particular in the third axial cutout and in the fourth axial cutout.

According to various embodiments, the intraocular lens has a further haptic arm, which is attached to the optical body, wherein the further haptic arm has a radial cutout formed in that side of the haptic arm arranged outwards in a further radial direction with respect to the optical axis. In particular, the further haptic arm can be configured with point symmetry in relation to the haptic arm. It is particularly preferred that the intraocular lens has a further medicament reservoir, which has the medicament and a through-hole through which the further haptic arm extends. Moreover, the further medicament reservoir is arranged in the radial cutout of the further haptic arm.

The medicament reservoir is preferably attached to the haptic arm by means of an interference fit. As a result, the medicament reservoir is arranged particularly firmly on the haptic arm.

According to various embodiments, the medicament reservoir is configured to dispense the medicament continuously. In particular, the medicament reservoir can be configured to be biodegraded. For this purpose, the medicament reservoir can have a matrix into which the medicament can be introduced, having a copolymer formed by a first monomer and a second monomer. The first monomer can be a caprolactone and the second monomer can be selected from the group of lactide, glycolide and/or trimethylene carbonate. An example of a matrix into which the medicament can be introduced, and which is not biodegradable, is a polymerized hydroxyethyl methacrylate.

The medicament can include, for example, an antibiotic such as moxifloxacin, and/or a steroidal anti-inflammatory agent such as dexamethasone, and/or a non-steroidal anti-inflammatory agent such as a non-steroidal anti-rheumatic agent, for example diclofenac. For example, the medicament can additionally or alternatively have a diagnostic substance, such as a contrast agent.

The medicament reservoir can, for example, have a single material having the matrix with the medicament introduced in the matrix or consisting of the matrix with the medicament introduced in the matrix.

The medicament reservoir preferably has a first material, having the matrix with the medicament introduced in the matrix, and a second material, which is different from the first material and forms the first web and in particular the second web. The second material is preferably not biodegradable. For this purpose, the second material can be selected from the group polymethyl methacrylate, polymerized hydroxyethyl methacrylate, polypropylene, silicone, acrylate copolymer.

### BRIEF DESCRIPTION OF DRAWINGS

The invention will now be described with reference to the drawings wherein:

FIG. 1 shows a plan view of a first intraocular lens according to the disclosure arranged in a capsular bag;

FIG. 2 shows a perspective view of a first medicament reservoir;

FIG. 3 shows a perspective view of a second medicament reservoir;

FIG. 4 shows a perspective view of a third medicament reservoir;

FIG. 5 shows a side view of a second intraocular lens according to the disclosure;

FIG. 6 shows a haptic arm and a medicament reservoir, the latter arranged spaced apart from the haptic arm;

FIG. 7 shows the haptic arm and the medicament reservoir from FIG. 6, with the medicament reservoir moved onto the haptic arm; and,

FIG. 8 shows the haptic arm and the medicament reservoir from FIGS. 6 and 7, which are arranged in a capsular bag, wherein a first web of the haptic arm is arranged in a first axial cutout of the haptic arm and a second web of the haptic arm is arranged in a second axial cutout of the haptic arm.

### DETAILED DESCRIPTION

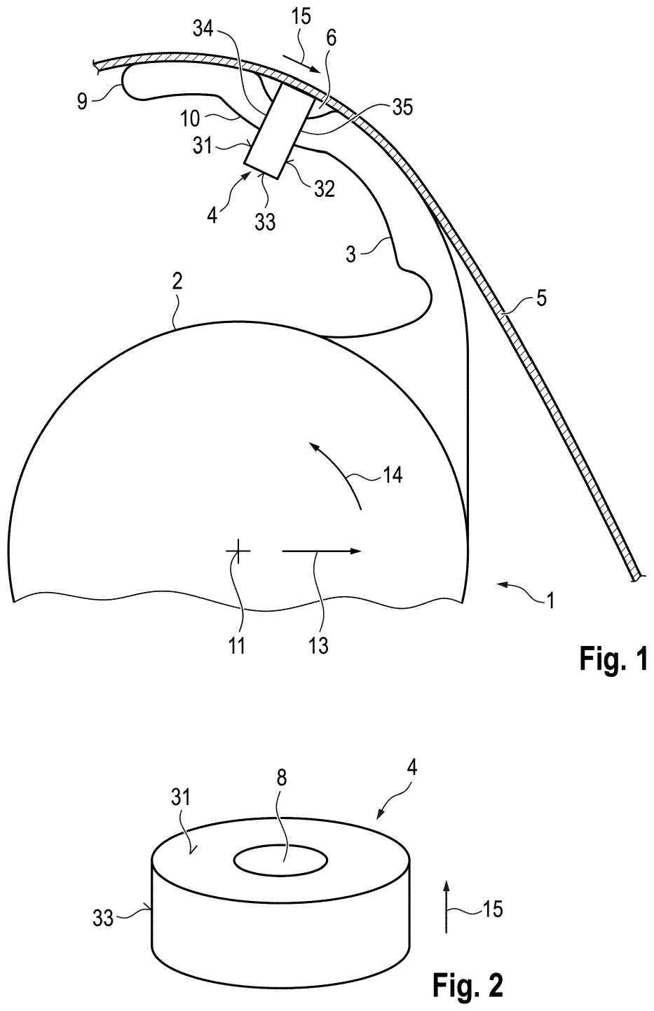

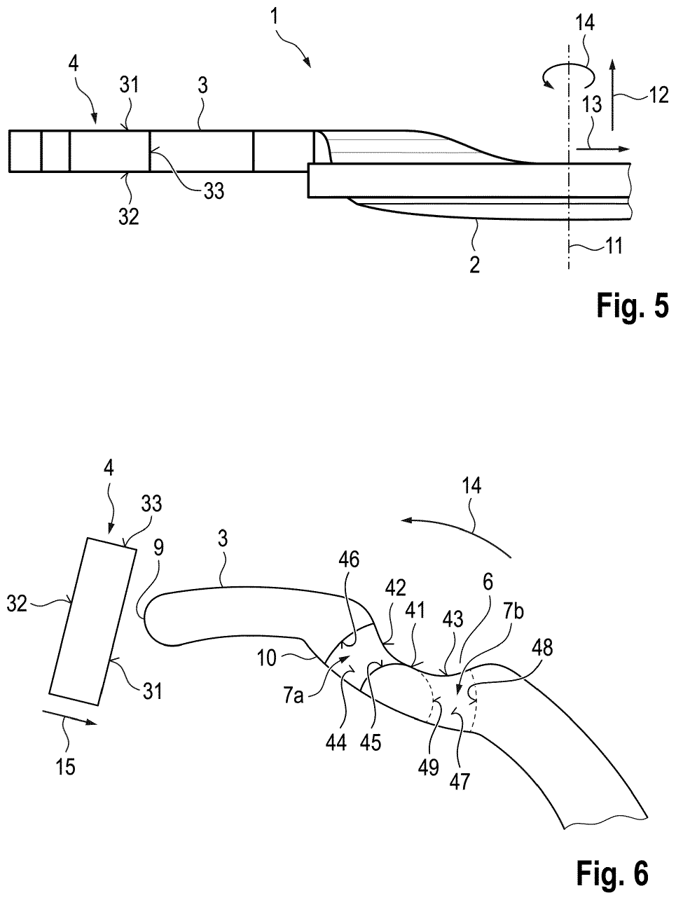

As can be seen from FIGS. 1 and 5, an intraocular lens 1 has an optical body 2, which has an optical axis 11, and a haptic arm 3, which is attached to the optical body 2. The haptic arm 3 has a radial cutout 6, which is configured to receive a medicament reservoir 4 and which is formed in a side of the haptic arm 3, which side is arranged outwards in a radial direction 13 with respect to the optical axis 11. The intraocular lens 1 can have a medicament reservoir 4, which has a medicament and is arranged in the radial cutout 6. The medicament reservoir 4 has a through-hole 8, through which the haptic arm 3 extends. The medicament reservoir 4 can be attached to the haptic arm 3, for example, by means of a positive fit and in particular by means of an interference fit. The haptic arm 3 can have a curved configuration, for example, and in particular can have a C-shaped (see FIG. 1) or J-shaped configuration.

FIGS. 1 to 8 show that the medicament reservoir 4 can have a first end face 31 and a second end face 32, which is arranged facing away from the first end face 31, wherein the through-hole 8 is limited by the first end face 31 and the second end face 32. The through-hole 8 has a first longitudinal end 34 in a region of the first end face 31 and a second longitudinal end 35 in a region of the second end face 32, wherein the medicament reservoir 4 has a displacement direction 15, which is directed from the first longitudinal end 34 to the second longitudinal end 35. The displacement direction 15 can be oriented in the direction in which the medicament reservoir 4 is to be displaced in order to move the medicament reservoir 4 into the radial cutout 6. It is conceivable that the displacement direction 15 is arranged parallel to a normal of the first end face 31 and/or parallel to a normal of the second end face 32. The first end face 31 can form the longitudinal end of the medicament reservoir 4 located in the displacement direction 15, and the second end face 32 can form the longitudinal end of the medicament reservoir 4 located in a direction which is oriented counter to the displacement direction 15. Moreover, on its outside, the medicament reservoir 4 can have a circumferential surface 33, which is arranged between the first end face 31 and the second end face 32 and in particular can directly adjoin the first end face 31 and the second end face 32.

FIG. 6 shows that the radial cutout 6 can be limited by a first flank 41, which limits a displacement of the medicament reservoir 4 counter to the radial direction 13, that is, towards the optical axis 11. Moreover, the radial cutout 6 can be limited by a second flank 42, which limits a displacement of the medicament reservoir 4 in a circumferential direction 14 with respect to the optical axis 11. In addition, the radial cutout 6 can be limited by a third flank 43, which limits a displacement of the medicament reservoir 4 in a direction that is oriented counter to the circumferential direction 14.

FIG. 2 shows a first embodiment for the medicament reservoir 4, in which the through-hole 8, at least at one point along the displacement direction 15, is limited about the entire circumference by the material of the medicament reservoir 4. It is also conceivable that the through-hole 8 is limited about the entire circumference by the material of the medicament reservoir 4 at each point along the displacement direction 15. Moreover, FIG. 2 shows that the through-hole 8, in a cross-sectional plane whose normal is parallel to the displacement direction 15, can have the shape of a circle. However, other shapes are also conceivable, for example an ellipse, a rectangle or a square. In addition, FIG. 2 shows that the circumferential surface 33 in the cross-sectional plane can have the shape of a circle. However, other shapes are also conceivable, for example an ellipse, a rectangle or a square. In the first embodiment of the medicament reservoir 4 (compare FIG. 2), it is conceivable that, when the medicament reservoir 4 is arranged in the radial cutout 6, the displacement direction 15 lies in a plane whose normal is arranged parallel to the optical axis 11 (compare FIG. 1). In the first embodiment of the medicament reservoir 4, it is moreover conceivable that the haptic arm 3 extends from the first longitudinal end 34 to the second longitudinal end 35.

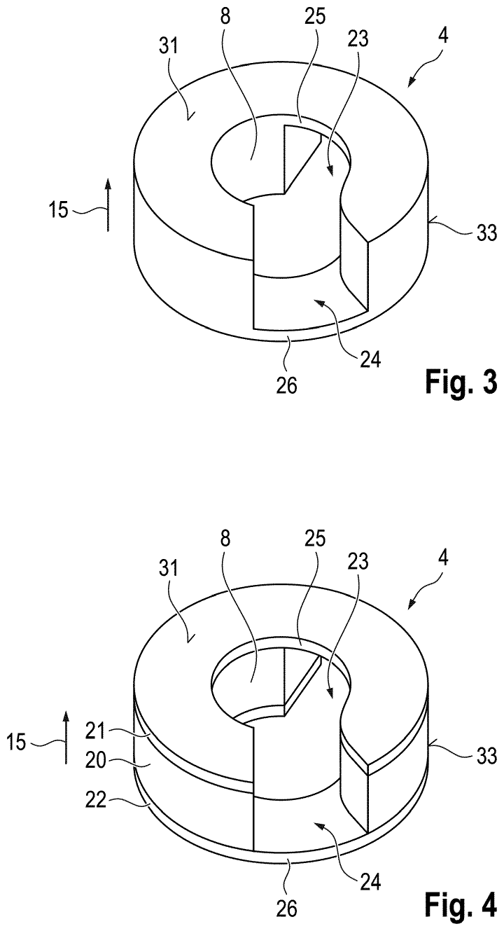

FIGS. 6 to 8 show that the haptic arm 3 can have a first axial cutout 7a, in which the medicament reservoir 4 is arranged and which is formed in a first side of the haptic arm 3, which side is arranged outwards in an axial direction 12 with respect to the optical axis 11. In this case, the medicament reservoir 4 can have a first medicament reservoir cutout 23 (compare the second embodiment of the medicament reservoir 4 in FIG. 3 and the third embodiment of the medicament reservoir 4 in FIG. 4), which communicates with the through-hole 8, and can have a first web 25, which limits the first medicament reservoir cutout 23, wherein the first web 25 is arranged in the first axial cutout 7a and the haptic arm 3 is arranged in the first medicament reservoir cutout 23. The first web 25 can form a part of the first end face 31, and the second end face 32 can be unformed in a region of the first medicament reservoir cutout 23 in order to allow insertion of the haptic arm 3 in the displacement direction 15 into the first medicament reservoir cutout 23.

It will also be seen from FIGS. 6 to 8 that the haptic arm 3 can have a second axial cutout 7b, in which the medicament reservoir 4 is arranged and which is arranged offset from the first axial cutout 7a in a circumferential direction 14 with respect to the optical axis 11 and is formed in a second side of the haptic arm 3, which side is arranged outwards counter to the axial direction 12 and is arranged facing away from the first side. In this case, the medicament reservoir 4 can have a second medicament reservoir cutout 24 (compare the second embodiment of the medicament reservoir 4 in FIG. 3 and the third embodiment of the medicament reservoir 4 in FIG. 4), which communicates with the through-hole 8, and a second web 26, which limits the second medicament reservoir cutout 24, wherein the second web 26 is arranged in the second axial cutout 7b and the haptic arm 3 is arranged in the second medicament reservoir cutout 24. The second web 26 can form a part of the second end face 32, and the first end face 31 can be unformed in a region of the second medicament reservoir cutout 24 in order to allow insertion of the haptic arm 3 counter to the displacement direction 15 into the second medicament reservoir cutout 24.

The first axial cutout 7a can directly adjoin the radial cutout 6, and/or the second axial cutout 7b can directly adjoin the radial cutout 6 (compare FIGS. 6 to 8). The second axial cutout 7b can be arranged spaced apart in the circumferential direction 14 from the first axial cutout 7a. FIGS. 1 and 6 to 8 show that the intraocular lens 1 can have a radial projection 10, which in the radial direction 13 protrudes inwards from the rest of the haptic arm 3 and which, in a circumferential direction 14 with respect to the optical axis 11, is arranged in the same region as the radial cutout 6.

The first web 25 can be recessed in the first axial cutout 7a, so that the medicament reservoir 4, seen in the radial direction 13, can be arranged in a region of the first side flush with the haptic arm 3 (compare FIG. 5). The second web 26 can be recessed in the second axial cutout 7b, so that the medicament reservoir 4, seen in the radial direction 13, can be arranged in a region of the second side flush with the haptic arm 3 (compare FIG. 5).

It is conceivable that, for the second embodiment of the medicament reservoir 4 and the third embodiment of the medicament reservoir 4, the displacement direction 15 is arranged parallel to the optical axis 11 (see FIG. 8).

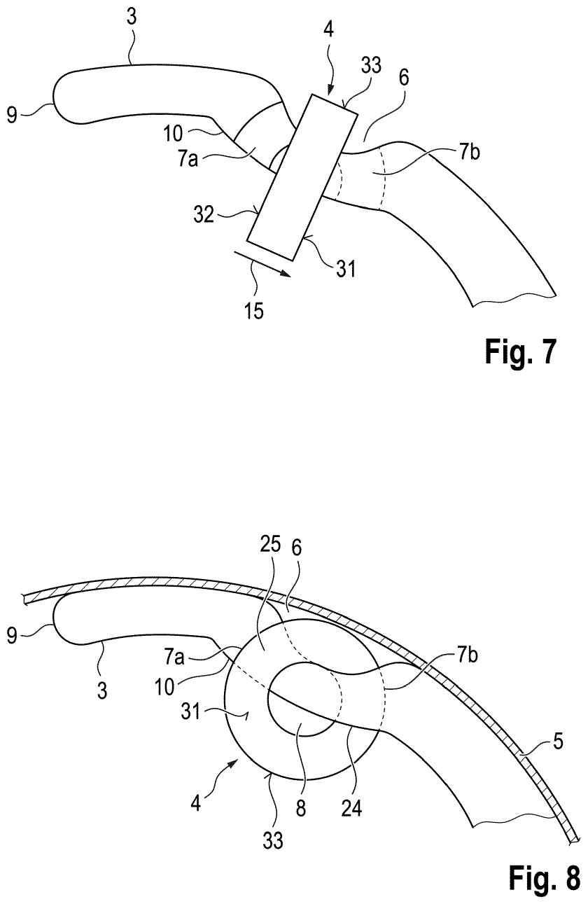

FIGS. 6 to 8 show how the medicament reservoir 4 can be introduced into the radial cutout 6. First, the medicament reservoir 4 is arranged spaced apart from the haptic arm 3. Subsequently, a longitudinal end 9 of the haptic arm 3, which end is arranged facing away from the optical body 2, can first be arranged in the through-hole 8. By a displacement of the medicament reservoir 4 in the displacement direction 15, the medicament reservoir 4 can be displaced until the medicament reservoir 4 reaches the radial cutout 6 (compare FIG. 7). In the first embodiment of the medicament reservoir 4, the displacement is completed here, and, for example, the arrangement shown in FIG. 1 is obtained. In the second and third embodiments of the medicament reservoir 4, a pivoting of the medicament reservoir 4 is still required so that the first web 25 enters the first axial cutout 7a and the second web 26 enters the second axial cutout 7b (compare FIG. 8).

FIG. 6 shows that the first axial cutout 7a can be limited by a first flank 44, which limits a displacement of the medicament reservoir 4 counter to the axial direction 12. Moreover, the first axial cutout 7a can be limited by a second flank 45, which limits a displacement of the medicament reservoir 4 counter to the circumferential direction 14. In addition, the first axial cutout 7a can be limited by a third flank 46, which limits a displacement of the medicament reservoir 4 in the circumferential direction 14. Moreover, FIG. 6 shows that the second axial cutout 7b can be limited by a first flank 47, which limits a displacement of the medicament reservoir 4 in the axial direction 12. Moreover, the second axial cutout 7b can be limited by a second flank 48, which limits a displacement of the medicament reservoir 4 counter to the circumferential direction 14. In addition, the second axial cutout 7b can be limited by a third flank 49, which limits a displacement of the medicament reservoir 4 in the circumferential direction 14.

The first flank 41 of the radial cutout 6 and the first flank 44 of the first axial cutout 7a can, for example, enclose an angle of 60° to 120°, in particular of 80° to 100°, or substantially 90°. The first flank 41 of the radial cutout 6 and the first flank 47 of the second axial cutout 7b can, for example, enclose an angle of 60° to 120°, in particular of 80° to 100°, or substantially 90°.

The medicament reservoir 4 can have only a single material, which has the medicament, as is the case in the second embodiment of the medicament reservoir 4 according to FIG. 3.

Alternatively, it is conceivable that the medicament reservoir 4 has a first material, which has the medicament, and a second material, which is different from the first material and forms the first web 25, as is the case in the third embodiment of the medicament reservoir 4 according to FIG. 4. For this purpose, the medicament reservoir 4 can have a first cover plate 21, which is formed of the second material and forms the first end face 31. It is also conceivable that the second material forms the second web 26. For this purpose, the medicament reservoir 4 can have a second cover plate 22, which is formed of the second material and forms the second end face 32. Between the first cover plate 21 and the second cover plate 22, a middle part 20 can be arranged which is formed of the first material and in particular forms the circumferential surface 33.

The first cover plate 21 and/or the second cover plate 22 can be porous. This allows the medicament to be released more quickly.

The medicament reservoir 4 can have a column extending through the first material and connecting the first cover plate 21 and the second cover plate 22 to each other. For example, the column can have the second material and/or consist of the second material. It is also conceivable to provide a plurality of the columns.

FIGS. 1 and 8 show that the medicament reservoir 4 can be recessed in the radial cutout 6. This has the effect that, when the intraocular lens 1 is inserted into the capsular bag 5 of an eye, the medicament reservoir 4 does not push the capsular bag 5 outwards, that is, away from the optical body 2 (compare FIGS. 1 and 8).

It is understood that the foregoing description is that of the preferred embodiments of the invention and that various changes and modifications may be made thereto without departing from the spirit and scope of the invention as defined in the appended claims.

### LIST OF REFERENCE SIGNS

1 intraocular lens
2 optical body
3 haptic arm
4 medicament reservoir
5 capsular bag
6 radial cutout
7a first axial cutout
7b second axial cutout
8 through-hole
9 longitudinal end
10 radial projection
11 optical axis
12 axial direction
13 radial direction
14 circumferential direction
15 displacement direction
20 middle part
21 first cover plate
22 second cover plate
23 first medicament reservoir cutout
24 second medicament reservoir cutout
25 first web
26 second web
31 first end face
32 second end face
33 circumferential surface
34 first longitudinal end
35 second longitudinal end
41 first flank of the radial cutout
42 second flank of the radial cutout
43 third flank of the radial cutout
44 first flank of the first axial cutout
45 second flank of the first axial cutout
46 third flank of the first axial cutout
47 first flank of the second axial cutout
48 second flank of the second axial cutout
49 third flank of the third axial cutout

1. An intraocular lens comprising:
an optical body defining an optical axis;
a haptic arm attached to said optical body;
said haptic arm having a radial cutout formed in a side of said haptic arm, wherein said side is arranged outwards in a radial direction with respect to said optical axis; and,
said haptic arm having a radial projection projecting inwards in the radial direction from said haptic arm and which, in a circumferential direction with respect to said optical axis, is arranged in a same region as said radial cutout.

2. The intraocular lens of claim 1, wherein said intraocular lens has a medicament reservoir which has a medicament and defines a through-hole in which said haptic arm is arranged; and, said medicament reservoir is arranged in said radial cutout.

3. The intraocular lens of claim 2, wherein said medicament reservoir is recessed in said radial cutout.

4. The intraocular lens of claim 1, wherein said haptic arm defines a first axial cutout formed in a first side of said haptic arm; and, said first side is arranged outwards in an axial direction with respect to said optical axis.

5. The intraocular lens of claim 2, wherein said haptic arm defines a first axial cutout formed in a first side of said haptic arm; said first side is arranged outwards in an axial direction with respect to said optical axis; and, said medicament reservoir is arranged in said first axial cutout.

6. The intraocular lens of claim 4, wherein said first axial cutout directly adjoins said radial cutout.

7. The intraocular lens of claim 4, wherein said haptic arm defines a second axial cutout arranged offset from said first axial cutout in the circumferential direction with respect to said optical axis and is formed in a second side of said haptic arm; and, said second side is arranged outwards counter to the axial direction and is arranged facing away from said first side.

8. The intraocular lens of claim 7, wherein said medicament reservoir is arranged in said second axial cutout.

9. The intraocular lens of claim 7, wherein said second axial cutout directly adjoins said radial cutout.

10. The intraocular lens of claim 7, wherein said second axial cutout is arranged spaced apart from said first axial cutout in the circumferential direction.

11. The intraocular lens of claim 1, wherein said haptic arm has a curved configuration.

12. The intraocular lens of claim 1, wherein said haptic arm has a C-shaped configuration or a J-shaped configuration.
